(19) United States
(12) Patent Application Publication
(10) Pub. No.: US 2005/0229828 A1
(43) Pub. Date: Oct. 20, 2005

(54) Method and business methods for applying biologically-processed mineral salts to crops and crop land

(76) Inventor: Kenneth R. Martin, Taylor, AZ (US)

Correspondence Address: MARTIN & ASSOCIATES, LLC, P O BOX 548, CARTHAGE, MO, 64836-0548, US

(21) Appl. No.: 10/827,707

(22) Filed: Apr. 20, 2004

(51) Int. Cl. A01C 1/00; A01H 9/00; A01H 11/00

(52) U.S. Cl. 111/200

(57) ABSTRACT

According to the preferred embodiments, mineral salts are added to an aqueous biological culture. The biological agents in the aqueous biological culture process one or more of the minerals into a different chemical form that is more readily assimilated by plant leaves and roots. The aqueous biological culture and processed mineral(s) are then applied to crops and/or crop land. The result is the easy application of minerals that have been biologically-processed in a manner that enhances their uptake by plant leaves and roots. The resulting plants provide crops that have enhanced concentrations of trace minerals, thereby providing enhanced health benefits to both animals and humans that consume the crops.

### BACKGROUND OF THE INVENTION

1. Technical Field

This invention generally relates to the field of agriculture, and more specifically relates to methods for treating crops and crop land.

2. Background Art

Mankind has used various techniques throughout the centuries to increase crop production. Early Indians on the North American continent buried a small fish when planting corn. Manure has been used extensively as fertilizer for a wide variety of crops. Modern advances have produced chemical fertilizers that are much more concentrated and much less expensive than naturally-occurring fertilizers. Most modern chemical fertilizers are characterized using three numbers that indicate the amount of Nitrogen, Phosphorous and Potassium, respectively, that is in the fertilizer on a percentage basis.

Dr. Maynard Murray performed several experiments over several decades in fertilizing crop land with dry mineral salts. His work is summarized in Maynard Murray, M.D., “Sea Energy Agriculture”, revised second edition, copyrights 1976 and 2003, available from Acres U.S.A., PO Box 91299, Austin, Tex. 78709, www.acresusa.com. Dr. Murray's book “Sea Energy Agriculture” is incorporated herein by reference. Dr. Murray states that minerals in soil have, over time, been leached out of the soil by rain water and have flowed into the oceans. He notes that chronic disease is not present in the ocean, and hypothesizes that this is due to the natural presence of a high number of trace elements (or minerals) in sea water. Dr. Murray experimented with applying dry sea salts to a variety of different crops, and had success in varying degrees with all the crops he treated.

Note for our discussion herein that the term “mineral” is used in its simplest sense to represent basic elements, but may also denote a combination of elements. For example, sea water contains relatively high concentrations of sodium chloride (NaCl), which is common salt. Sodium is an element, and is therefore a mineral in its simplest stated. Similarly, chlorine is an element, and is likewise a mineral in its simplest state. However, salt is also a mineral, even though the elements of sodium and chlorine that make up a salt molecule are also minerals in their most basic sense. The term “mineral” is used herein to denote any basic element or molecule that is capable of being biologically-processed.

Referring to FIG. 1, a method 100 in accordance with the teachings of Dr. Murray's book begins by evaporating water from ocean water, thereby producing dry mineral salts (step 110). The dry mineral salts are then applied to crop land (step 120). Crops are raised on the crop land treated in step 120, and are then harvested (step 130). Note that the dry mineral salts could be applied either before the crop is planted, or after the crop is planted. Dr. Murray's work did not end with the application of dry mineral salts to crop land. He went on to experiment with feeding animals with feed raised on land that had the dry sea salts applied. This is shown in step 140 of FIG. 2. Dr. Murray documented significant differences between animals that were fed feed made of grain raised on land treated with the dry mineral salts and animals in the control group that were fed the same feed mix made from grains raised on land not treated with the dry mineral salts.

A company known as OceanGrown, LLC, P.O. Box 458, Ft. Myers, Fla. 33902 has built upon Dr. Murray's original work in treating crop land with ocean minerals. The OceanGrown product is known as OceanSolution, which is a liquid form of sea water that has been processed in some way. OceanGrown claims that its manner of making OceanSolution is proprietary information, but does state that the processing is done without heat or chemical processing, and without using chelates. A prior art process 300 in accordance with the OceanGrown product and method is shown in FIG. 3. First, ocean water is processed (step 310). The processed ocean water is then applied to crop land (step 320). Crops are then raised and harvested from the crop land (step 330).

Both Dr. Murray's work in applying dry mineral salts to crop land and OceanGrown's work in applying processed sea water to crop land are both based on the premise that crops (plants) can only use minerals in an inorganic state. According to both Dr. Murray and OceanGrown, the plants take in the inorganic minerals, and add a carbon atom to the minerals to put them in an organic state that is readily used by and beneficial to animals and humans. However, the exact mechanisms that govern mineral assimilation are not completely understood, and the assumption that plants can only absorb inorganic minerals may be incorrect. What is needed is a way to provide minerals in an organic state that may be more readily absorbed by the leaves and roots of plants.

### DISCLOSURE OF INVENTION

According to the preferred embodiments, mineral salts are added to an aqueous biological culture. The biological agents in the aqueous biological culture process one or more of the minerals into a different chemical form that is more readily assimilated by plant leaves and roots. The aqueous biological culture and processed mineral(s) are then applied to crops and/or crop land. The result is the easy application of minerals that have been biologically-processed in a manner that enhances their uptake by plant leaves and roots. The resulting plants provide crops that have enhanced concentrations of trace minerals, thereby providing enhanced health benefits to both animals and humans that consume the crops.

The foregoing and other features and advantages of the invention will be apparent from the following more particular description of preferred embodiments of the invention, as illustrated in the accompanying drawings.

### BRIEF DESCRIPTION OF DRAWINGS

The preferred embodiments of the present invention will hereinafter be described in conjunction with the appended drawings, where like designations denote like elements, and:

FIG. 1 is a flow diagram of a prior art method for applying dry mineral salts to crop land;

FIG. 2 is a flow diagram of a prior art method for feeding the crops raised in method 100 of FIG. 1 to animals;

FIG. 3 is a flow diagram of a prior art method for applying processed ocean water to crop land;

FIG. 4 is a flow diagram of a prior art method for applying an aqueous biological culture to crops and crop land;

FIG. 5 is a block diagram showing a prior art system that could be used in performing method 400 of FIG. 4;

FIG. 6 is a flow diagram of a first prior art way of applying the aqueous biological culture to crops and crop land in step 420 of FIG. 4;

FIG. 7 is a flow diagram of a second prior art way of applying the aqueous biological culture to crops and crop land in step 420 of FIG. 4;

FIG. 8 is a flow diagram of a third prior art way of applying the aqueous biological culture to crops and crop land in step 420 of FIG. 4;

FIG. 9 is a flow diagram of a prior art method for doing business;

FIG. 10 is a flow diagram of a method in accordance with the preferred embodiments;

FIG. 11 is a flow diagram of a method for a farmer to do business in accordance with method 1000 in FIG. 10;

FIG. 12 is a block diagram of a system in accordance with the preferred embodiments that could be used in performing method 1100 of FIG. 11;

FIG. 13 is a method for doing business in accordance with the preferred embodiments; and

FIG. 14 is a method for a farmer to do business in accordance with the preferred embodiments.

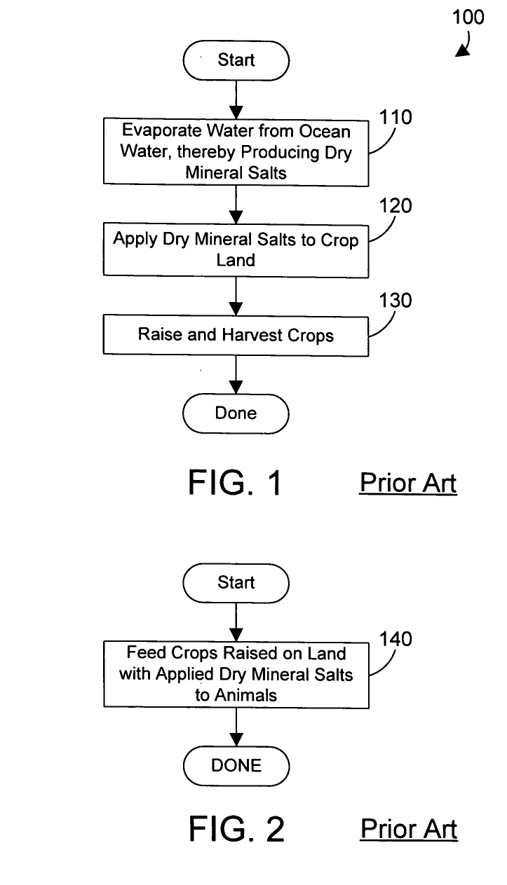

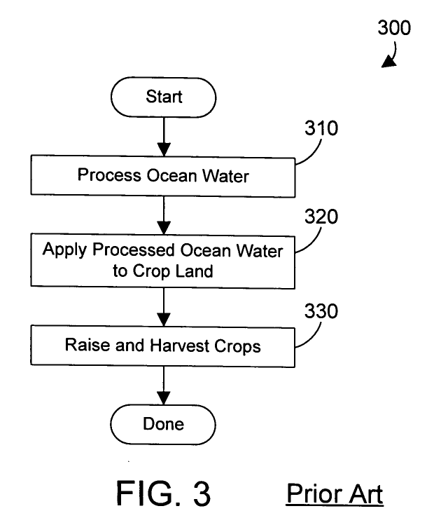

### BEST MODE FOR CARRYING OUT THE INVENTION

Overview

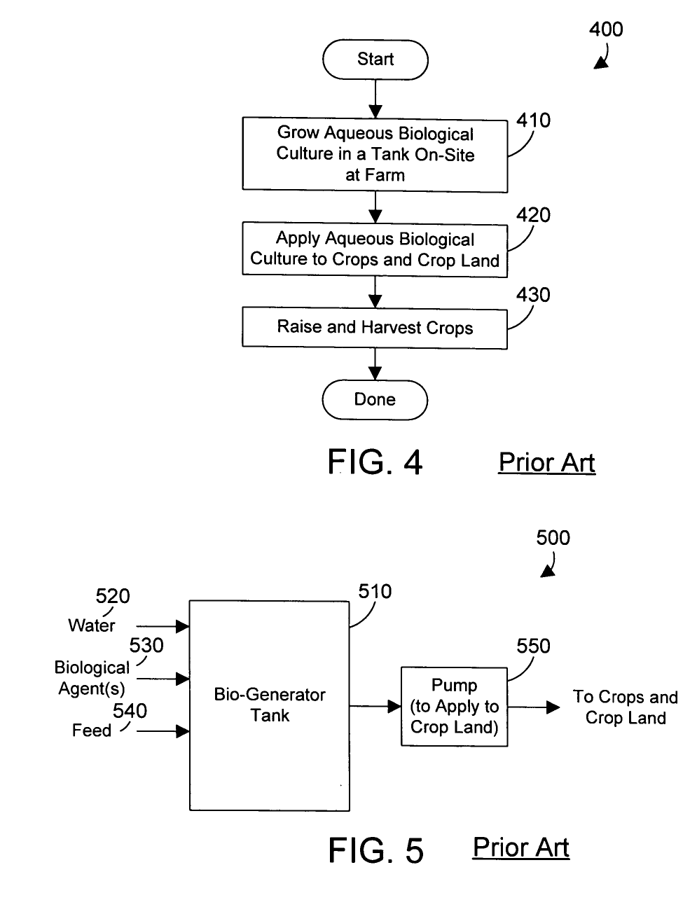

Work has been done in other fields that are not related in any way to the application of mineral salts to farm land. For example, Martin BioChem, Inc., 1776 W. Rutledge Dr., Snowflake, Ariz. 85937 provides a service to farmers that includes growing a biological culture on-site at a farm for application to crops and crop land. A method 400 shown in FIG. 4 represents a method for a farmer to do business using the service provided by Martin BioChem, Inc. First, an aqueous biological culture is grown in a tank on-site at a farm (step 410). The aqueous biological culture is then applied to crops and crop land (step 420). Crops are then raised and harvested from the crop land (step 430). The biological culture used in the past by Martin BioChem is an algae/bacteria culture that enhances biological activity in the soil. Note that the algae/bacterial culture may be applied not only to crop land, but also to crops. It is believed that plants take in some nutrients via their leaves in addition to their roots. The concept of “foliar feeding” is known in the art, and refers to the placement of nutrients on the leaves (or foliage) of a plant, which are then absorbed by some degree by the plant leaves.

Referring to FIG. 5, a prior art system 500 for the Martin BioChem method 400 in FIG. 4 includes a Bio-Generator tank 510. A Bio-Generator tank 510 may be any suitable tank that is capable of holding liquid. In the preferred implementation, Bio-Generator tank 510 is a cattle watering tank or an above-ground swimming pool. Water 520, biological agent(s) 530, and feed 540 are added to the Bio-Generator tank 510, which causes the desired biological culture to grow. The Martin BioChem biological agents comprise a mixture of algae and bacteria. The feed 540 has organic acids, carbohydrates, and other nutrients.

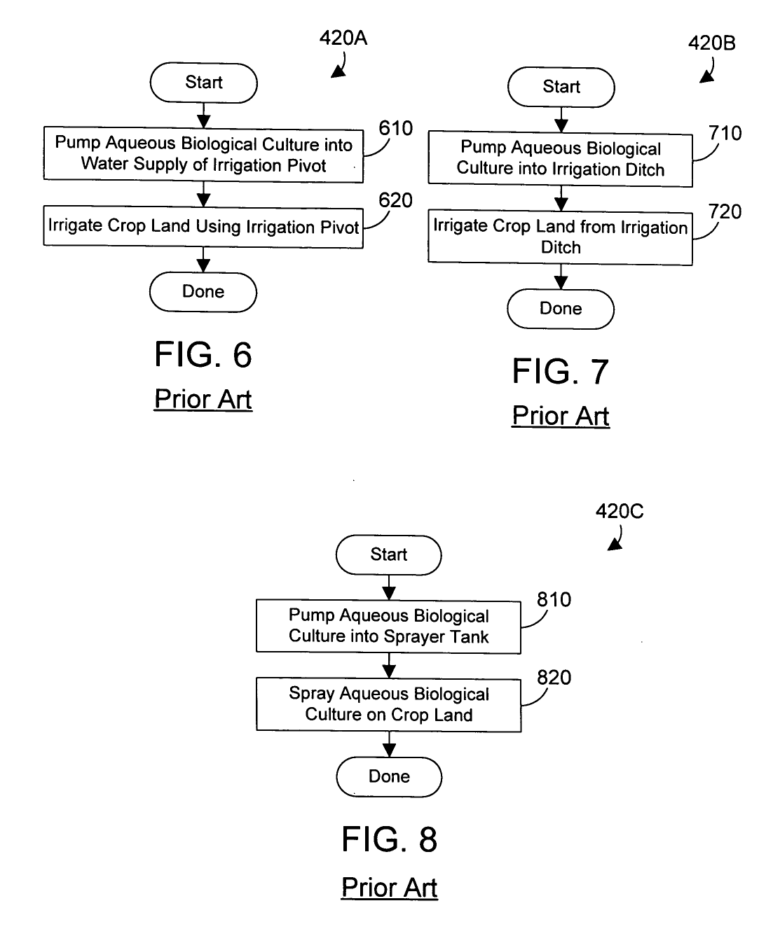

Once the desired biological culture is achieved, the aqueous biological culture is pumped via pump 550 to be applied to crops and crop land. Note that pump 550 may deliver the aqueous biological culture to different destinations depending on the type of distribution that is setup on the farm. For example, FIGS. 6-8 show different ways of applying the aqueous biological culture to crops and crop land in step 420 in FIG. 4. A first method 420A in FIG. 6 pumps the aqueous biological culture 610 into the water supply of an irrigation pivot (step 610). The crop land is then irrigated using the irrigation pivot (step 620). Irrigation pivots are well-known in the Western United States. An irrigation pivot is a large sprinkler system that has a central connection point and a long arm that pivots about the central connection point. The pivot arm includes sprinkler heads that apply water to the crops and crop land below. Wheels allow the pivot arm to move in a circle, thereby providing irrigation water to a circular crop area.

A second way that the Martin BioChem aqueous biological culture has been applied to crops and crop land is via irrigation ditches. Referring to FIG. 7, a second method 420B for applying aqueous biological culture to crop land pumps the aqueous biological culture into an irrigation ditch (step 710). Crop land is then irrigated from the irrigation ditch (step 720). A third way that the Martin BioChem aqueous biological culture has been applied to crops and crop land is via a vehicle that has a sprayer tank. Referring to FIG. 8, a third method 420C for applying the aqueous biological culture to crops and crop land pumps the aqueous biological culture into a sprayer tank on a vehicle (step 810). The vehicle then sprays the aqueous biological culture from the sprayer tank onto crops and crop land (step 820). The Martin BioChem aqueous biological culture has a high concentration of biological components that allow the culture to be diluted with relatively large amounts of water. Methods 420A and 420B in FIGS. 6 and 7 allow diluting the culture with a relatively large quantity of water. Method 420C in FIG. 8 allows controlling the concentration applied according to the spray head(s) used and the speed of travel of the vehicle. In addition, the aqueous biological culture could be diluted with a suitable amount of water or other liquid, as required, to achieve a desired dosage rate application on the crops and crop land.

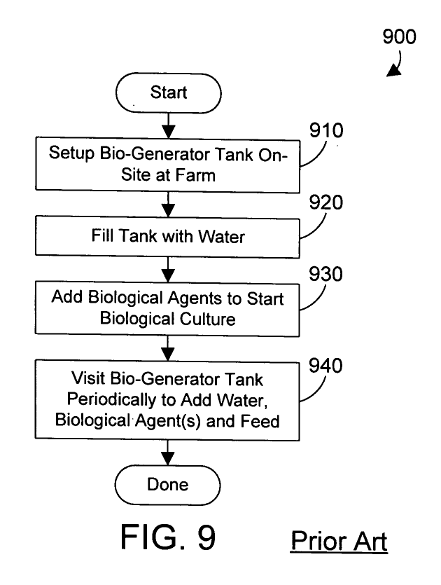

A known method for doing business is shown as method 900 in FIG. 9. Method 900 represents a method that Martin BioChem, Inc. has used. First, a Bio-Generator tank is setup on-site at the farm (step 910). The tank is filled with water (step 920). Biological agents are then added to the water to start the biological culture (step 930). The biological agents for Martin BioChem are concentrated algae/bacteria cultures. The tank is then left for some period of time until the biological culture achieves its desired concentration. In the case of Martin BioChem, sunlight accelerates the growth of the algae/bacteria culture. The farmer can then pump the aqueous biological culture out of the Bio-Generator tank for distribution on the crops and crop land. Note that only a portion of the aqueous biological culture is removed from the Bio-Generator tank at one time, allowing the aqueous biological culture to regenerate and thereby provide a perpetual supply of aqueous biological culture. A person that is a field representative of Martin BioChem visits the Bio-Generator tank periodically, and adds water, biological agents, and feed into the Bio-Generator tank (step 940) to maintain the aqueous biological culture in a strong and growing state. Note that the term “feed” is used in a broad sense herein to mean anything that may be consumed or digested by the biological culture. In the case of the Martin BioChem algae/bacteria culture, the preferred feed includes organic acids, carbohydrates (such as molasses), and other nutrients. The method 900 for doing business is thus a service that Martin BioChem provides to farmers that provides the farmer a perpetual supply of aqueous biological culture on-site for ready application to the crops and crop land at the farmer's convenience whenever crops or crop land are irrigated.

### Description of the Preferred Embodiments

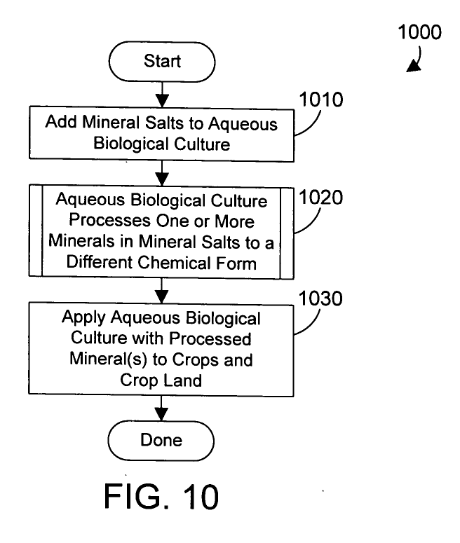

Referring now to FIG. 10, a method 1000 in accordance with the preferred embodiments begins by adding mineral salts to an aqueous biological culture (step 1010). Note that the term “aqueous biological culture” simply means a culture of live biological agents, such as bacteria and algae, that are grown in an environment that is mostly water. Because the biological culture in step 1010 contains live biological agents, it may be properly referred to as an “active” biological culture. The aqueous biological culture then processes one or more minerals in the mineral salts to a different chemical form (step 1020). The aqueous biological culture with the processed mineral(s) is then applied to crops and crop land (step 1030).

The biological processing of one or more minerals to a different chemical form in step 1020 may be accomplished using a number of different biological agents that have different biological effects on the mineral(s). For example, commercially-available bacteria may be used to chelate one or more of the minerals. Chelation is a process whereby a metal ion is firmly bound with an organic molecule (ligand) to form a ring structure. The resulting ring structure protects the mineral from entering into unwanted chemical reactions. An algae culture could be used as the biological agent, which also chelates one or more minerals in the mineral salts. Other biological agents could include protozoa and fungi. Note that the biological agents themselves may not perform the transformation to a different chemical form, but may create enzymes that transform the minerals to a different chemical form. In addition, some limited testing by C. Louis Kervran has shown that it may be possible to achieve biological transmutation of one element to a different element. In such tests, sodium was apparently converted to potassium. The preferred embodiments expressly extend to the use of any biological agent that is capable of processing one or more minerals to a different chemical form.

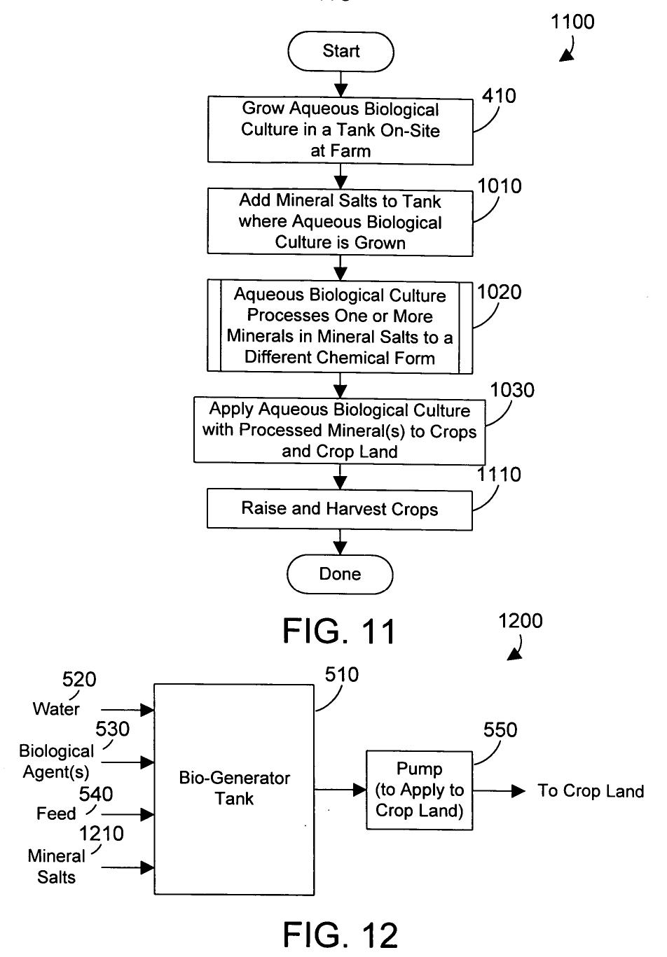

Referring to FIG. 11, a method 1100 for doing business in accordance with the preferred embodiments begins by growing an aqueous biological culture in a tank on-site at a farm (step 410). Mineral salts are then added to the aqueous biological culture in the tank (step 1010). The aqueous biological culture then processes one or more minerals in the mineral salts to a different chemical form (step 1020). The aqueous biological culture with the processed mineral(s) is then applied to crop land (step 1030). Crops are then raised and harvested on the crop land treated in step 1030 (step 1110).

A system 1200 in accordance with the preferred embodiments includes many of the same features 510-550 shown in FIG. 5. Note, however, that system 1200 additionally includes the addition of mineral salts 1210 to the Bio-Generator tank 510. The addition of the mineral salts 1210 to the Bio-Generator tank 510 allows the biological agents 530 to process one or more of the minerals in the mineral salts 1210 to a different chemical form.

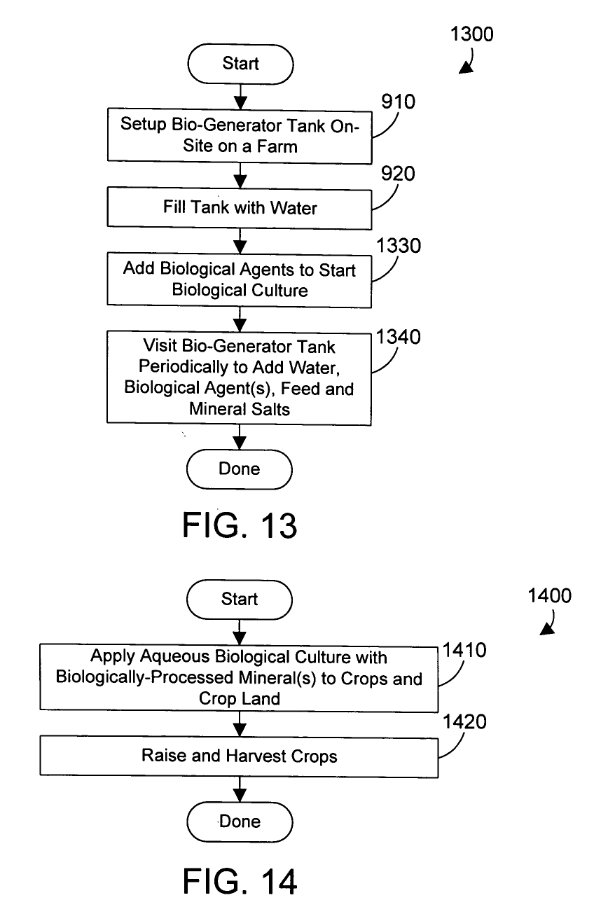

Referring to FIG. 13, a method 1300 for doing business in accordance with the preferred embodiments begins by setting up a Bio-Generator tank on-site on a farm (step 910). The tank is filled with water (step 920). Biological agents are then added to the water in the tank to start a biological culture (step 1330). At this point the biological culture is allowed to grow in the tank until it reaches a desired concentration level. The last step is to periodically visit the Bio-Generator tank to add water, one or more biological agents, feed, and mineral salts (step 1340). We note that method 1300 varies from prior art method 900 in FIG. 9 by the type of biological agents added in step 1330 and by the addition of mineral salts to the biological culture. By selecting biological agents that efficiently process minerals, and by adding the mineral salts to the aqueous biological culture that includes these specially-selected biological agents, the aqueous biological culture processes one or more of the minerals in the mineral salts to a different chemical form.

One of the significant advantages of method 1300 in FIG. 13 is that the same delivery systems used to distribute the aqueous biological culture in the prior art may also be used to distribute the aqueous biological culture that includes one or more biologically-processed minerals. A field representative that is already on-site to service a tank that contains algae/bacteria culture may also service a tank with the aqueous biological culture that biologically processes minerals without making an extra trip to the farm. Thus, significant value is added by the field representative servicing two tanks without incurring significant additional cost, thereby creating additional profit opportunity for the company that practices method 1300 in FIG. 13.

In the most preferred implementation, two Bio-Generator tanks may be setup, one to grow the Martin BioChem algae/bacteria culture, and a second to grow the aqueous biological culture of the preferred embodiments that biologically processes one or more minerals. Of course, a single Bio-Generator tank could also be used that does not include the Martin BioChem algae/bacteria culture, but instead uses off-the-shelf biological agents, as discussed in more detail below.

Referring to FIG. 14, a method 1400 for a farmer to do business includes the steps of applying an aqueous biological culture with biologically-processed mineral(s) to crop land (step 1410), and raising and harvesting crops grown on the treated crop land (step 1420). Method 1400 is a way for a farmer to make a large number of trace minerals available in an organic form for absorption by crops, thereby making the crops more nutritious due to the presence of additional trace minerals. These minerals enhance food for better flavor, better shelf life, and increased health for the consumer. In modern agriculture, it is well-recognized by many scientists that food grown with the present chemical fertilizer technology has lower mineral and nutritional levels that in the past. It is believed that the use of biologically-processed minerals as outlined herein will contribute significantly to improved health of humans and other animals who consume food from crops that have higher mineral content due to the application of the biologically-processed minerals to the crops and crop land.

The methods of the preferred embodiments differ significantly from the earlier work done by Dr. Murray and OceanGrown. The premise underlying the research of Dr. Murray and OceanGrown is that plants can only absorb inorganic forms of minerals. Once the inorganic forms of minerals are absorbed by the roots of a plant, the plant adds a carbon atom, convert these inorganic forms of minerals to an organic form, which can then be readily used when ingested by animals or humans. This premise may be correct, but incomplete. While it is true that a plant may be able to absorb some amount of inorganic minerals directly from the soil, it is believed that the rate and efficiency of absorption by the plant leaves and roots are greatly enhanced if the mineral is provided to the leaves and roots of the plant in an organic form. Thus, by processing inorganic minerals using an active biological culture, the inorganic minerals are converted to an organic form before the minerals are made available to the plants. The organic form of minerals that have been processed by the active biological culture is more readily absorbed by the plant leaves and roots, resulting in more efficient absorption of the minerals by the plants. The work of Dr. Murray and OceanGrown thus expressly teach away from biologically processing mineral salts to an organic form before application to the plant, because Dr. Murray and OceanGrown base their research and products on the premise that only inorganic minerals may be absorbed by plants.

The concept of biologically processing minerals is in its infancy, and much research is needed to determine optimal concentrations of minerals, optimal types and concentrations of biological agents, and optimal time periods for biologically processing the mineral salts by the biological agents. However, at this early stage of the game, with no field testing to provide empirical results, it is believed that the invention is enabled and a best mode is provided as detailed below.

We assume the Bio-Generator tank is a stock tank such as those used to water cattle, with a capacity of 650 gallons (2,460 liters). The tank is filled with fresh water. The bacterial agent of choice is a product called Spectrum Extra that is available from Tanio Technology and Technique, Inc., S. 12102 Andrus Rd, Cheney, Wash. 99004. Spectrum Extra is a dry power that contains bacteria that have been specially design to digest minerals. A dose of 0.9 dry ounces (25 grams) of Spectrum Extra is added to the water in the tank. Six fluid ounces (180 ml) of a product known as Pepzyme G is then added to the tank. Pepzyme G contains enzymes in liquid form, and is available from Tanio Technology and Technique, Inc. Enzymes are organic catalysts which assist in developing an environment more conducive to mineral digestion by the bacteria. The bacteria also produce enzymes in the process of chelating the minerals. Note that the addition of enzymes in the preferred embodiments is not required, but helps to speed the biological processing of minerals in the mineral salts. One quart (0.95 liter) of molasses is also added to the tank. The molasses provides feed in the form of carbohydrates that the bacteria may use as a source of fuel. A suitable mineral salt is a product known as Redmond RealSalt that is available from Redmond Minerals, Inc., 6005 N. 100 W., PO Box 219, Redmond, Utah 84652. Redmond RealSalt is a deposit of sea salt in the Utah desert. Two hundred pounds (91 kg) of Redmond RealSalt are added to the tank. The contents of the tank are then stirred together to assure all of the additives are well-mixed and to assure all of the minerals have fully dissolved. The mixing may be done manually using a large stirring device such as a canoe paddle, but is preferably done using a pump that includes a vacuum wand to recirculate mixture in the tank until all of the granular minerals are dissolved. The aqueous biological culture is then left to grow. At this point, the way the aqueous biological culture is distributed determines what happens next. When the aqueous biological culture is to be applied to crops and crop land via an irrigation pivot, an injector pump transfers approximately 7.5 gallons (28.4 liters) per hour into the irrigation pivot, resulting in a total of 180 gallons (682 liters) per day of aqueous biological culture being removed from the tank. An automatic water filling device with a float will add water to the tank to maintained the desired level of liquid in the tank, resulting in an inflow of water into the tank at the same rate that the aqueous biological culture is removed. The result is a continuous, perpetual process where a very large quantity of aqueous biological culture may be provided to the farmer over a period of time.

When the aqueous biological culture is to be applied to crops and crop land via irrigation ditches, a water inlet valve on one side of the tank is used that has a controlled flow rate of approximately 7.5 gallons (28.4 liters) per hour, resulting in 180 gallons (682 liters) per day. An overflow outlet on the opposite side of the tank allows the aqueous biological culture to spill out of the tank into an irrigation ditch as the water is added. This results in approximately 180 gallons (682 liters) of aqueous biological culture being spilled out of the overflow outlet into the irrigation ditch per day. Again, this is a continuous process, providing a steady supply of aqueous biological culture to the farmer.

When the aqueous biological culture is to be applied to crops and crop land via a vehicle-propelled sprayer tank, the same water inlet valve described above is used that has a controlled flow rate of approximately 7.5 gallons (28.4 liters) per hour, resulting in 180 gallons (682 liters) per day. The difference between the irrigation ditch case described above and the sprayer case described here is that the overflow outlet now dumps into a second tank. The aqueous biological culture may then be pumped from the second tank into a larger holding tank, or directly into a sprayer tank. In all three cases, a continuous supply of aqueous biological solution is provided to the farmer over an extended period of time, because the aqueous biological culture is able to regenerate itself provided that the rate of removal of aqueous biological culture and rate of adding water are controlled to assure the aqueous biological culture remains alive and thriving.

After removing the 180 gallons (682 liters) per day of aqueous biological culture and replacing this with fresh water for seven days, a field service person then returns to the Bio-Generator tank to add Spectrum Extra, Pepzyme G, molasses, and Redmond RealSalt in the same proportions as before. The result is an aqueous biological culture that contains biologically-processed mineral salts that may be provided to a farmer in a perpetual manner. Note that at the end of each week, the Spectrum Extra, Pepzyme G, molasses, and Redmond RealSalt in the specified proportions are preferably added to the existing aqueous biological culture. However, it is equally within the scope of the preferred embodiments to empty the tank and start over each week.

Another method within the scope of the preferred embodiments provides the biological agents and feed using a metered application system to distribute these items into the Bio-Generator tank from a jug. For example, the proportions of Spectrum Extra, Pepzyme G, and molasses could be added to a jug of water and mixed thoroughly. A metering pump could then dispense a small amount from the jug into the Bio-Generator tank at periodic intervals, thereby providing a continuous feed of these items into the Bio-Generator tank. Note that the mineral salts are still added directly into the Bio-Generator tank and stirred until dissolved. When this type of system is used, the field service person returns periodically to mix the minerals in the Bio-Generator tank and to refill the jug so the system can continue to automatically feed the mixture from the jug into the Bio-Generator tank.

Let's assume the 650 gallon (2,460 liter) tank described above provides its output to an irrigation pivot that irrigates a 120 acre (49 hectare) field. Let's further assume that the quantities listed above are added to the tank every week for 12 weeks. The result is that 2400 lbs (1090 kg) of minerals that are biologically-processed will be applied to the 120 acres (49 hectares) of land, resulting in a net application rate of 20 lb per acre (22 kg per hectare). It is believed that 20 lb per acre (22 kg per hectares) will provide significant advantages compared to untreated crop land. Note, however, that the rate of applying the mineral salts may go up or down depending on the economics of the crops being raised. For a high-value crop like strawberries, a much higher application rate may be used, as high as 1000 lb per acre (1120 kg per hectare). For lower-value crops such as barley or wheat, the 20 lb per acre (22 kg per hectare) rate will provide significant results at reasonable cost. The application rate of the mineral salts to crop land may vary from 1 lb per acre (1.12 kg per hectare) to 1000 lb per acre (1120 kg per hectare) within the scope of the preferred embodiments.

Note that the proportions of ingredients above indicate the best mode of the invention, but the invention may also be practiced using variations in the quantities of these ingredients. For example, the amount of Spectrum Extra could vary from 0.35 dry ounces (10 grams) to 3.5 dry ounces (100 grams). The amount of Pepzyme G could vary from 3 fluid ounces (89 ml) to 32 fluid ounces (0.95 liter). The amount of molasses could vary from one pint (0.47 liter) to one gallon (3.8 liters). The amount of mineral salts could vary from 10 lbs (4.5 kg) to 1000 pounds (450 kg).

The concentration of the mineral salts in the aqueous biological culture may be measured to assure adequate concentration of minerals. In the most preferred implementation, the total dissolved solids (TDS) is from 10,000 to 50,000 parts per million (PPM). However, the TDS may vary from 2,000 to 100,000 PPM within the scope of the preferred embodiments, depending on the variation in the quantity of mineral salts and water that are actually used.

The Redmond RealSalt that is the preferred form of mineral salts is approximately 98% sodium chloride (NaCl), with the remaining portion made up of a large number of trace elements. Note, however, that other types of mineral salts may be used within the scope of the preferred embodiments. These mineral salts may be naturally-occurring, or may be manufactured. The preferred embodiments preferably use a mineral salt that is at least 50% NaCl, with the remaining portion including at least the following elements: calcium, potassium, sulfur, magnesium, and phosphorous. The preferred embodiments more preferably use a mineral salt that contains at least 95% NaCl, and the remaining part is at least fifty elements that are naturally-occurring in sea water. The preferred embodiments most preferably use a mineral salt that contains at least 97% NaCl, and the remaining part is over seventy elements that are naturally-occurring in sea water. The OceanGrown web site lists a total of 95 elements that are naturally-occurring in sea water that Dr. Murray considered to be essential to plant, animal and human health. These elements include: Hydrogen, Helium, Lithium, Beryllium, Boron, Carbon, Nitrogen, Oxygen, Fluorine, Neon, Sodium, Magnesium, Aluminum, Silicon, Phosphorus, Sulfur, Chlorine, Argon, Potassium, Calcium, Scandium, Titanium, Vanadium, Chromium, Manganese, Iron, Cobalt, Nickel, Copper, Zinc, Gallium, Germanium, Arsenic, Selenium, Bromine, Krypton, Rubidium, Strontium, Yttrium, Zirconium, Niobium, Molybdenum, Technetium, Ruthenium, Rhodium, Palladium, Silver, Cadmium, Indium, Tin, Antimony, Tellurium, Iodine, Xenon, Cesium, Barium, Lanthanum, Cerium, Praseodymium, Neodymium, Promethium, Samarium, Europium, Gadolinium, Terbium, Dysprosium, Holmium, Erbium. Thulium, Ytterbium, Lutetium, Hafnium, Tantalum, Tungsten, Rhenium, Osmium, Iridium, Platinum, Gold, Mercury, Thallium, Lead, Bismuth, Polonium, Astatine, Radon, Francium, Radium, Actinium, Thorium, Protactinium, Uranium, Neptunium, Plutonium, and Americium.

One of the significant advantages of the preferred embodiments is the ability to make a relatively large number of trace minerals available to plants in an organic form that is more readily absorbed by plant leaves and roots. The biological processing of mineral salts in accordance with the preferred embodiments has the capability of simultaneously processing a relatively large number of minerals in the mineral salts to different chemical forms. These different chemical forms may include organic forms that are more readily absorbed by plant leaves and roots.

One skilled in the art will appreciate that many variations are possible within the scope of the present invention. Thus, while the invention has been particularly shown and described with reference to preferred embodiments thereof, it will be understood by those skilled in the art that these and other changes in form and details may be made therein without departing from the spirit and scope of the invention.

1. A method for farming comprising:
(1) applying an aqueous biological culture containing at least one biologically-processed mineral to crop land, wherein the at least one biologically-processed mineral is biologically-processed from mineral salt that comprise at least 50% sodium chloride, with the remaining portion of the mineral salt comprising at least the following elements:
calcium;
potassium;
sulfur;
magnesium; and
phosphorous;
(2) raising a crop on the crop land; and
(3) harvesting the crop.

2. The method for farming of claim 1 further comprising the step of applying the aqueous biological culture to the crop grown on the crop land.

3. The method for farming of claim 1 wherein the biologically-processed mineral comprises an inorganic mineral that is biologically-processed by at least one biological agent in the aqueous biological culture.

4. The method for farming of claim 3 wherein the at least one biological agent chelates at least one mineral in the mineral salt.

5. The method for farming of claim 1 wherein the mineral salt comprises at least fifty minerals that are naturally-occurring in sea water.

6. The method for farming of claim 1 wherein the mineral salt comprises over seventy minerals that are naturally-occurring in sea water.

7. A method for doing business comprising the steps of:
adding a mineral salt to an aqueous biological culture;
the aqueous biological culture biologically processing the mineral salt to a different chemical form; and
applying the aqueous biological culture with biologically-processed minerals to crop land.

8. The method for doing business of claim 7 further comprising the step of applying the aqueous biological culture to at least one crop grown on the crop land.

9. The method for doing business of claim 7 wherein the aqueous biological culture chelates at least one mineral in the mineral salt.

10. The method for doing business of claim 7 wherein the mineral salt comprises an inorganic mineral.

11. The method for doing business of claim 7 wherein the mineral salt comprises at least fifty minerals that are naturally-occurring in sea water.

12. The method for doing business of claim 7 wherein the mineral salt comprises over seventy minerals that are naturally-occurring in sea water.

13. The method for doing business of claim 7 wherein the mineral salt comprises at least 50% sodium chloride, with the remaining portion of the mineral salt comprising at least the following elements:
calcium;
potassium;
sulfur;
magnesium; and
phosphorous.

14. A method for doing business comprising the steps of:
setting up a bio-generator tank on-site at a farm;
filling the bio-generator tank with water;
adding at least one biological agent to the water in the bio-generator tank; and
periodically visiting the bio-generator tank to add:
water;
at least one biological agent;
feed for the at least one biological agent; and
mineral salt.

15. The method for doing business of claim 14 wherein the at least one biological agent comprises at least one bacteria that is capable of digesting the mineral salt.

16. The method for doing business of claim 14 wherein the at least one biological agent chelates at least one mineral in the mineral salt.

17. The method for doing business of claim 14 wherein the feed comprises a source of carbohydrates.

18. The method for doing business of claim 14 wherein the mineral salt is inorganic.

19. The method for doing business of claim 14 wherein the mineral salt comprises at least fifty minerals that are naturally-occurring in sea water.

20. The method for doing business of claim 14 wherein the mineral salt comprises over seventy minerals that are naturally-occurring in sea water.

21. The method for doing business of claim 14 wherein the mineral salt comprises at least 50% sodium chloride, with the remaining portion of the mineral salt comprising at least the following elements:
calcium;
potassium;
sulfur;
magnesium; and
phosphorous.

22. A method for biologically processing an inorganic mineral in a mineral salt into an organic form, the method comprising the steps of:
generating an aqueous active biological culture;
adding the inorganic mineral to the aqueous active biological culture; and
the aqueous active biological culture biologically processing the inorganic material into the organic form.

23. The method of claim 22 wherein the aqueous active biological culture chelates the inorganic mineral.

24. The method of claim 22 wherein the inorganic mineral comprises at least fifty minerals that are naturally-occurring in sea water.

25. The method of claim 22 wherein the inorganic mineral comprises over seventy minerals that are naturally-occurring in sea water.

26. The method of claim 22 wherein the mineral salt comprises at least 50% sodium chloride, with the remaining portion of the mineral salt comprising at least the following elements:
calcium;
potassium;
sulfur;
magnesium; and
phosphorous.

27. A method for changing chemical form of a mineral from a first form to a second form, the method comprising the steps of:
generating an aqueous active biological culture;
adding the mineral of the first form to the aqueous active biological culture; and
the aqueous active biological culture biologically processing the mineral of the first form to the second form.

28. The method of claim 27 wherein the first form comprises an inorganic form and the second form comprises an organic form.

29. The method of claim 27 wherein the aqueous active biological culture chelates the mineral of the first form to generate therefrom the second form.

30. The method of claim 27 wherein the mineral comprises at least fifty minerals that are naturally-occurring in sea water.

31. The method of claim 27 wherein the inorganic mineral comprises over seventy minerals that are naturally-occurring in sea water.

32. The method of claim 27 wherein the mineral salt comprises at least 50% sodium chloride, with the remaining portion of the mineral salt comprising at least the following elements:
calcium;
potassium;
sulfur;
magnesium; and
phosphorous.
